(19) United States
(12) Patent Application Publication
(10) Pub. No.: US 2010/0113965 A1
(43) Pub. Date: May 6, 2010

(54) INTERNAL SYSTEMS IMBALANCE DETERMINATION AND ITS USE FOR SELECTION OF FOOD SUPPLEMENTS AND/OR VITAMINS

(75) Inventors: Alexander Kanevsky, Arad (IL); Elena Kanevsky, Arad (IL); Haim Straze, Arad (IL); Ilia Kreiman, Beer Sheva (IL)

Correspondence Address: THE NATH LAW GROUP, 112 South West Street, Alexandria, VA, 22314, US

(73) Assignee: MEDEX SCREEN LTD., Arad (IL)

(21) Appl. No.: 12/531,997

(22) PCT Filed: Mar. 20, 2008
(86) PCT No.: PCT/IL08/00395

§ 371 Date: Jan. 10, 2010

Related U.S. Application Data
(60) Provisional application No. 60/907,112, filed on Mar. 21, 2007.

(51) Int. Cl. A61B 5/053

(52) U.S. Cl. 600/547

(57) ABSTRACT

An internal systems imbalance determining method for use in the determination of suitable food supplements provision for a patient, is provided. The patient has on his skin at least one sensing zone with at least one measurement point located therein, and at least one stimulation point. The method includes providing a diagnostic device for measuring electrical resistance between the measurement point on one side of the body, and a ground point on the other side of the body; providing at least one positioning member adapted to be placed over and at least partially cover the at least one sensing zone of the patient; placing the positioning member on the sensing zone on one side of the body; bringing the active electrode in contact with the measurement point through the positioning hole to provide the electrical signal thereto and providing electrical ground at the ground point by the dual-purpose electrode, and collecting a first measurement therefrom; removing the active electrode and electrically stimulating the body at the stimulation point using the dual-purpose electrode; repeating the above to collect a second measurement from the measurement point; performing analysis of the first and the second measurements thereby allocating possible imbalance of internal systems of the body; and outputting results of the analysis.

### FIELD OF THE INVENTION

This invention relates to diagnostic systems.

### BACKGROUND OF THE INVENTION

It is known that there are points on the skin of the human body at which the electric resistance may be different from that of the surrounding area, for example as a result of some actual or potential pathological phenomena. It has been found that each one of these points can be related to a particular organ of the body.

The points of the body at which the electrical resistance is measured on the skin are referred to as Biologically Active Points (hereinafter BAPs, or BAP in singular). These points are usually located in dermal-visceral zones (DVZ's), which have a higher electrical sensitivity than other skin zones of the body. It has also been found that there are twin-meridians in a human body, one for the left side of the body, and one for the right, and that each meridian has a corresponding BAP that represents it and provides an average value of electrical resistance therefore. Thus, twin-meridians have corresponding twin BAPs.

In U.S. Pat. No. 6,934,581, the entire content of which is incorporated herein by reference, there is described in detail a method for utilizing such BAPs as source-points, announcement points, sympathetic points and energy reference points for assessing the physiological condition of a diagnosed person.

Briefly, according to U.S. Pat. No. 6,934,581, a number of BAPs are selected on a body and the skin resistance at those points is measured twice to form two sets of results. The first set of measurement results includes the skin resistance at the BAPs without stimulating the body, whereas the second set comprises measurement results of skin resistance at the same BAPs after stimulating the body (the stimulation will be described below). Then, a normal corridor is conventionally constructed (sometimes referred to as a universal corridor) according to the results of the measurements, and if both a specific result from the first set of measurements (i.e., before applying stimulation to the body) and a corresponding result from the second set of measurements (i.e., after stimulating the body) fall outside the normal corridor, these two specific results indicate the presence of a disease in the corresponding organ. However, if one of the results in one of the two sets of measurements falls inside the normal corridor, then if the corresponding result from the other set of measurements falls outside the universal corridor, it is considered a false disease indication and is therefore disregarded.

In other words, if there is a measurement result, either from the first or from the second set of measurements, that exceeds the universal corridor (hereinafter, “a meaningful result”), which can potentially indicate an infected organ, this meaningful result might be rendered useless by a corresponding result from the other set of measurements should the latter result lie within the universal corridor. A measurement result that lies within the universal corridor may be referred to as a “concealed result”.

According to U.S. Pat. No. 6,934,581, two sets of measurements are compared, e.g., by superimposing them on one another, for example on a computer monitor, and diagnostic conclusions are reached based on the comparison. However, in some cases, one or more of the measured values, which can belong to the first, second or both sets of measurements, become concealed after being superimposed on one another, because the concealed measurement resides entirely within the universal corridor. In such cases, no decisive medical decision can be made with respect to the organ whose BAP measurement value is concealed.

The stimulations referred to above may be of the type referred to as “electropunctural stimulation” or “under-load probing”, or transcutaneous electrical nerve stimulation (hereinafter “TENS”). It is known that the stimulation is particularly effective when applied to E36 BAPs on the legs and GI 4 BAPs on the arms, as their stimulation makes all systems in the body more active simultaneously, and these points are thus referred to as “general influence points”. Electrical stimulation is generally applied by increasing a current applied to the skin, usually from 0 through a maximum of about 25 mA, until a sensation of tingling is felt by the user. At this level of stimulation, a stimulation countdown timer is initiated.

The purpose of the stimulation is to make the patient's system of homeostasis more active. Homeostasis is the system which regulates the production and the action (influence) on the target organs, of hormones, enzymes, amines. It regulates the blood circulation, the immune system's functioning, nerve-muscle connections, and metabolic processes. The doctrine of homeostasis is at the basis of conventional ideas about man's normal and pathological physiology; it constitutes its main section. In other words, homeostasis is the state of all the processes of the organism being balanced. When homeostasis is disturbed, it is an indication that a disease has set in.

The system of homeostasis reacts to a certain extent to all the external and internal influences, like changes in the weather, acclimatizing, physical or psychological stress, alcohol consumption, food consumption, intake of medicine, physiotherapy, any organ's pathology, etc. Under the influence of TENS stimulation, defensive response reactions of the homeostasis system become active.

In WO 2005/117522, the entire content of which is incorporated herein by reference, there is disclosed a method of modifying the corridor such as to make concealed measurements available to a therapist, to allow him to consider every measurement and, thus, to obtain more accurate conclusions regarding problematic organs of the monitored person.

### SUMMARY OF THE INVENTION

According to one aspect of the present invention there is provided an internal systems imbalance determining method, and a system for performing it, for use in the determination of suitable food supplements and vitamins provision for, and in the control of their usage by, a patient; the patient having on his skin at least one sensing zone with at least one measurement point located therein, and at least one stimulation point, said method comprising:
(a) providing a diagnostic device for measuring electrical resistance between said at least one measurement point on one side of the patient's body, and a ground point on the other side of the body which is electrically connected to ground, by applying electrical signal to said at least one measurement point, said device comprising an active electrode adapted to provide said electrical signal by coming in contact with said at least one measurement point, and at least one dual-purpose electrode adapted to selectively provide electrical ground or stimulation when in contact with a corresponding ground or stimulation point on the patient's skin;
(b) providing at least one positioning member adapted to be placed over and at least partially cover said at least one sensing zone of the patient, and formed with at least one positioning hole adapted to coincide with said at least one measurement point, when the positioning member is placed over said at least one sensing zone, so as to allocate said at least one measurement point and allow said active electrode to come in contact therewith through said at least one positioning hole;
(c) placing said positioning member on said sensing zone on one side of the body;
(d) bringing said active electrode in contact with said measurement point through said positioning hole, to provide said electrical signal thereto, and providing electrical ground at said ground point by said dual-purpose electrode, and collecting a first measurement therefrom;
(e) removing said active electrode and providing electrical stimulation the body at said stimulation point using said dual-purpose electrode;
(f) repeating step (d) for collecting a second measurement from said measurement point;
(g) performing analysis of said first and said second measurements thereby allocating possible imbalance of internal systems of the body; and
(h) outputting results of said analysis in a manner suitable, or presenting recommendations suitable for selecting at least one food supplement and/or vitamin, based on said analysis.

The term ‘stimulation point’ used in the present description and claims means a zone on a patient's skin where this stimulation may be achieved. For simplification of the method, the stimulation point may also be used as ground point.

The above method may allow an individual not skilled in the medical arts to perform the above steps, without prior medical knowledge.

The measuring in steps (c) and (e) above, may be performed at a plurality of measurement points on different sides of the patient's body, and the stimulation in step (e) above, may accordingly be performed at a corresponding plurality of stimulation points.

The measurement points may be any of those disposed at dermal-visceral zones (DVZs) and, particularly, may be any of BAPs as described in the Background of the Invention, e.g. source-points, announcement points, sympathetic points and energy reference points.

The measurements may, for example, be taken from four BAPs on each leg and arm, summing up to sixteen measurements altogether, forming a set, whereby there is provided in step (d) above, a first set of measurements prior to stimulation, and in step (f) above a second set of measurements after stimulation. The BAPs from which measurements are taken may be the following points (eight on each side of the patient): P9, MC7, C7, GI5, RP3, F3, V65 and E42, corresponding to the internal systems of the body. The electrical signal provided by the active electrode for the purpose of said measurements may be between 0 and 20 μA.

The diagnostic device may be adapted to provide an assessment of internal systems conditions in order to determine body imbalance based on the sets of said measurements, said internal systems being one or more of the following: Immune, Endocrine, Respiratory, Cardiovascular, Digestive and Genital-Urinary.

The diagnostic device may also be provided with software adapted to output a body balance report with results indicating possible qualitative and quantitative imbalance in any one of the above systems, as previously described. The diagnostic device may also be adapted for providing, based on said imbalance, an output with a detailed recommendation as to preferred supplements and vitamins required for amendment thereof.

The results may be transferred to a computer so that a patient may keep track of his state when performing the diagnosis over a period of time. The results may be transferred to the patient for his reference via e-mail or may even be burned to a CD or DVD. This may allow the patient to keep track not only after his physical state but also on his food supplement and vitamins diet, as well as its effects on his state.

The diagnostic device may comprise four dual purpose electrodes each for attachment to one of four stimulating points, which may be such BAPs as E36 on the left leg, E36 on the right leg, GI 4 on the left arm, and GI 4 on the right arm.

Each of the dual-purpose electrodes may be in the form of a pad attachable to the patient's skin in a removable manner and connectable to the device diagnostic via an electric cable. The electrode is adapted, in its stimulating mode, to apply stimulation by electrical current which may be between 0 and 25 mA, and may be in the form of a pulsed current of a frequency substantially in the range of 75 to 125 Hz, for example substantially 100 Hz. The pulses may be rectangular pulses, and may provide a voltage in the range of 3 to 5 volts.

According to a second aspect of the present invention there is provided a positioning member for use with a diagnostic device having an electrode, and adapted for mounting on a patient, the patient having on his skin at least one sensing zone with at least one measurement point located therein, said positioning member being adapted to be placed over and at least partially cover said sensing zone, and being formed with at least one positioning hole adapted to coincide with said at least one measurement point, when the positioning member is placed over the sensing zone, so as to allocate said at least one measurement point and allow said electrode to come in contact therewith through said at least one positioning hole.

In accordance with a still further aspect of the invention, there is provided a package for use with a diagnostic device of the kind described above, comprising a plurality of positioning members as defined above, which may be of one or different types depending on the location of measuring points with which the electrode of said device will need to contact.

The positioning member may be in the form of a strap, band, or the like, and may be adapted to be placed on a wrist or a foot of the patient.

In particular, positioning members may be provided, adapted for mounting on a left foot, a right foot, a left hand or a right hand. Consequently, there may be provided a single orientation positioning member, specifically designed for a left or a right orientation. More particularly, a right hand positioning member will not fit a left hand. Alternatively, there may be provided a dual orientation positioning member adapted to switch between a left and a right orientation, for example, by inverting it inside out. This way a left oriented foot positioning member may be similarly used for the right foot and a left oriented hand positioning member may be similarly used for the right hand. In this case, the positioning member may be fitted with a securing mechanism adapted to keep the positioning member attached to the patient's body in both orientations.

The positioning members may be sold in packages including positioning members of the same orientation or kits of members with different orientations. Such a kit may comprise four single orientation positioning members adapted to be placed on the right and left hand, and the right and left foot. Alternatively, such a kit may comprise two dual orientation positioning members, e.g. one for the left and right hand, and one for the left and right foot.

Each positioning member may be formed with four positioning holes, coinciding with four BAPs disposed along the wrist or foot. The positioning member may also be constructed so as not to bias the measurements taken by the diagnostic device and may be made of simple disposable materials such as fabric, plastic etc.

Each positioning hole in the positioning member may be marked, or have an indicia, so as to indicate the appropriate BAP with which it is to be associated for taking measurement therein, to indicate to the operator the order of BAPs at which measurements are to be taken, as well as associate the measurement acquired with the appropriate BAP.

The present invention is particularly advantageous for determining desirable food supplements based on a 16-points diagnostic method which in accordance with a still further aspect of the invention comprises an algorithm adapted to use the required number of measurement points in order to produce a diagnosis evaluating the functioning of internal systems, said method comprising:
a. gathering measurements from the above sixteen BAPs mentioned above before and after a stimulation;
b. constructing a normal corridor based on said measurements using a midline as described in WO 2005/117522;
c. determining the degree of functioning of one of the aforementioned six internal systems according to the degree of deviation from said normal corridor of the measurements at the points where each one of said sixteen measurements has been taken.

In accordance with a still further aspect of the invention, there is provided a diagnostic device for the implementation of the 16-points diagnostic method.

### BRIEF DESCRIPTION OF THE DRAWINGS

In order to understand the invention and to see how it may be carried out in practice, an embodiment will now be described, by way of non-limiting example only, with reference to the accompanying drawings, in which:

FIG. 1 is a table showing the location of measurement points on a left foot and a left hand;

FIGS. 2A and 2B are enlarged schematic views of a left hand and a left foot with the measurements points of FIG. 1;

FIGS. 3A and 3B are schematic views of a left hand and a left leg with stimulation points thereon;

FIGS. 4A and 4B are schematic views of positioning members according to an embodiment of the present invention, adapted for mounting on the hand and the foot shown in FIGS. 2A and 2B;

FIGS. 5A and 5B are schematic views of the positioning members shown in FIGS. 4A and 4B, attached to the hand and the foot shown in FIGS. 2A and 2B;

FIG. 6 is a schematic view of a diagnostic device which may be used to perform measurements in accordance with an embodiment of the present invention; and

FIG. 7 is a schematic diagram of an exemplary report which may be provided in accordance with an embodiment of the present invention.

### DETAILED DESCRIPTION OF EXEMPLARY EMBODIMENTS

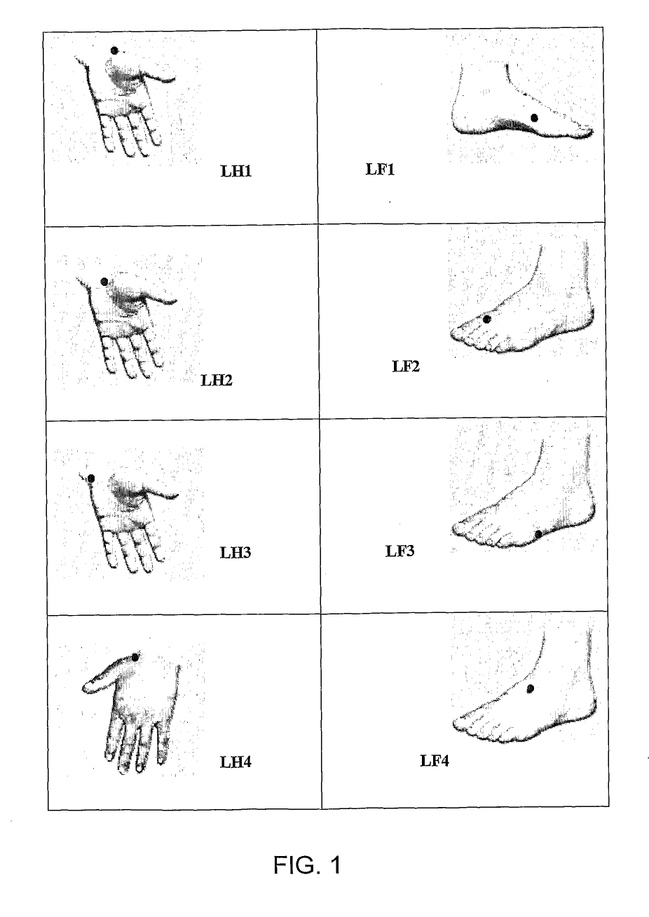

Referring to FIG. 1, the location of four measurement points is shown for each hand and each foot for a patient. Point LH1 to LH4 represent BAPs P9, MC7, C7 and GI5 respectively on the left hand, and points LF1 to LF4 represent BAPs RP3, F3, V65 and E42 respectively on the left foot. The following table specifies internal systems of a patient and their associated BAPs for each measuring point shown in FIG. 1.

| Measurement point | BAP | Corresponding System |
|---|---|---|
| LH1 | P9 | Respiratory |
| LH2 | MC7 | Cardiovascular |
| LH3 | C7 | Cardiovascular |
| LH4 | GI5 | Digestive |
| LF1 | RP3 | Immune + Endocrine |
| LF2 | F3 | Digestive |
| LF3 | V65 | Genitourinary |
| LF4 | E42 | Digestive |

It should be noted that while reference is made here to measurement points on the left hand and foot, identical points are found on the right hand and foot, similarly referred to as RH1 to RH4 and RF1 to RF4.

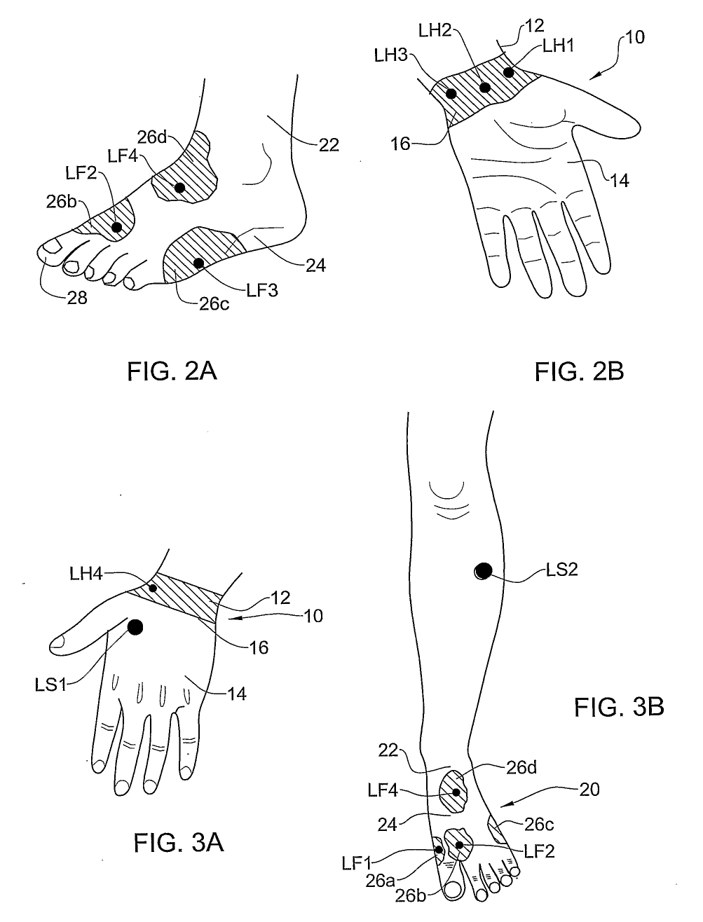

Referring to FIGS. 2A and 2B, a hand 10 having a wrist section 12 and a palm section 14, and a foot 20 having an ankle section 22 and a foot section 24 are shown. The hand 10 has a sensing zone 16 located on the wrist section 12 containing measurement points H1, H2, H3 and H4 (not shown). The foot 20 has, on the foot section 24, four sensing zones: 26a (not shown) located on the left side of the foot 20, 26b located near the bog toe 28, 26c located on the right side of the foot 20 and 26d, located on the ridge of the foot 20. Measurement points LF1 to LF4 are located within sensing zones 26a to 26d respectively.

FIGS. 3A and 3B demonstrate the location of two stimulation points, LS1 on the left hand and LS2 on the left leg, corresponding to BAPs GI 4 and E36 respectively. The stimulation points are located outside the sensing zones 16 and 26 for reasons that will become evident later.

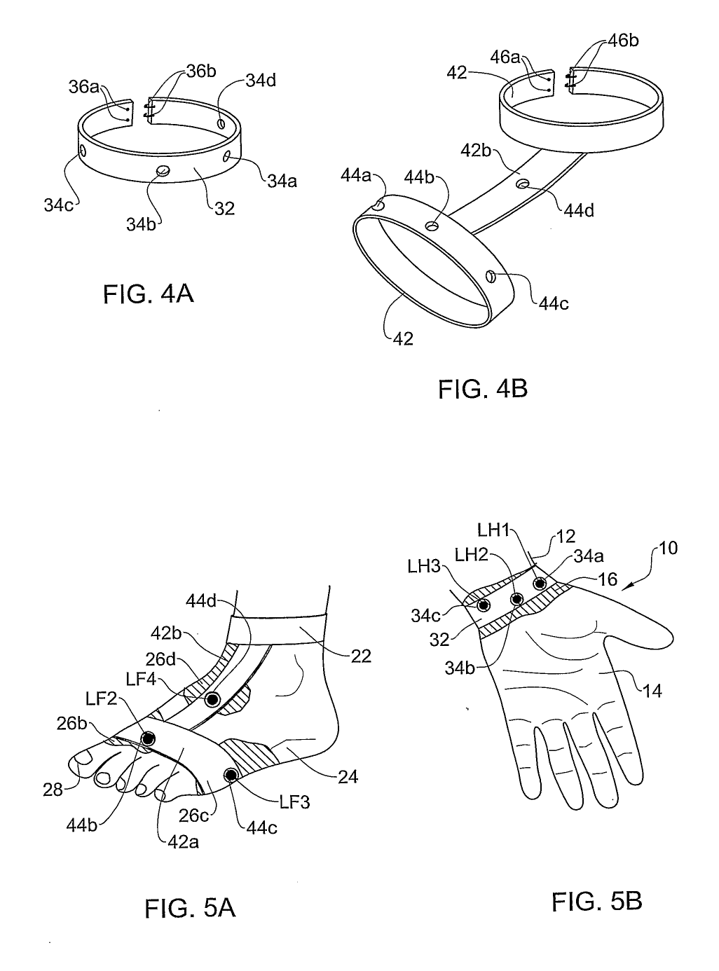

Turning to FIG. 4A, a hand positioning member 30 in the form of a wrist strap 32 is shown. The wrist strap 32 is formed with four positioning holes 34a to 34d, corresponding to measurement points LH1 to LH4. The wrist strap 32 is adapted to be fastened around the wrist section 12 of the hand 10 by a loop 36a and hook 36b mechanism.

Referring to FIG. 4B, the foot positioning member 40 comprises a toe strap 42a, a connecting strap 42b, and an ankle strap 42c. The toe strap and connecting strap are formed with four positioning holes 44a to 44d, corresponding to measurement points LF1 to LF4. The ankle strap 42c is fastened, much like the wrist 32 by a loop 46a and hook 46b mechanism.

FIG. 5A shows the hand positioning member 30 when placed on the hand 10. The wrist strap 32 is placed on the wrist section 12 of the hand 10, in such a way that it partially covers the sensing zone 16. In this position, the positioning holes 34a to 34d coincide with the measurement points LH1 to LH4, allowing them to be easily allocated when necessary. It should be noted here, that allocation of the measurements points without the positioning member 30, requires extensive training, which is normally provided by a course or a number of practice sessions. The same is true for measurement points on the foot, as described below.

With reference to FIG. 5B, the foot positioning member 40 is placed so that the toe strap 42a surrounds the foot 10 just above the toes, and the ankle strap 42c is fastened around the ankle 22, and the connecting strap 42b lies flat against the upper ridge of the foot section 24. In this position, the positioning holes 44a to 44d coincide with the measurement points LF1 to LF4, allowing their easy allocation when necessary.

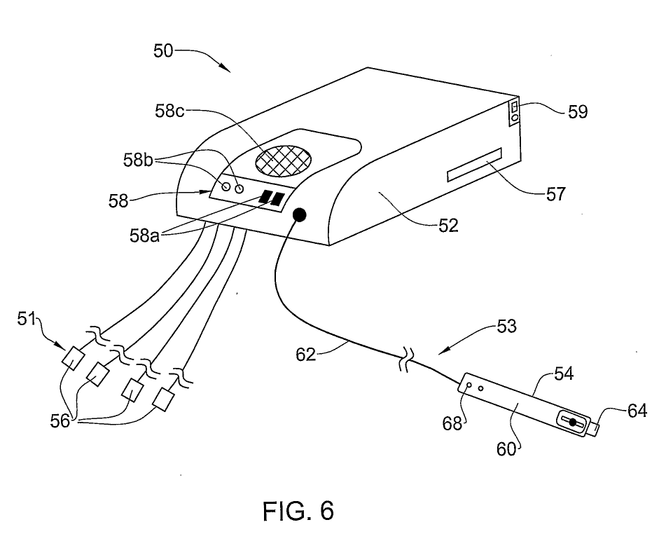

FIG. 6 illustrates a diagnostic device, generally indicated at 50, adapted for use for taking measurements at the measurement points referred above, for drawing a diagnostic conclusion for a patient suitable for making recommendations on food supplements and/or vitamins to be taken by the patient. The device comprises a main housing 52, a signal system 53 adapted to deliver an electrical signal, a stimulation system 51 adapted for selectively delivering a stimulation to the patient or constituting an electrical ground, and a user interface 58. A processing unit (not shown), in communication with each of the above, is provided within the housing 52. The signal system 53 comprises at least one active electrode 54 connected to an electrical signal generator (not shown). The active electrode 54 may be in the form of an electrode probe, or in the form of a plurality of pads detachably attachable to the patient's skin. The stimulation system 51 comprises a stimulation generator (not shown), a controller (not shown), and four dual-purpose electrodes 56. The controller is electrically connected both the stimulation generator and to electrical ground. In addition, the device 50 may comprise a removable media reader/writer 57, such as a disk drive, and an external data interface 59, such as a data port, through which data, for example measurement or analysis results, is adapted to be outputted.

The device 50 is adapted to operate in one of two operative states. In the first operative state, the device allows a user to measure the electrical resistance on a point or points of a patient's skin. In this state, the dual-purpose electrodes 56 are in their grounding mode of operation, wherein the controller selectively connects one of the dual-purpose electrodes 56 to electrical ground. In this way, should the active electrode 54 contact a BAP on the patient's skin, the resistance across the body will be accurately measured. In the second operative state of the device 50, the device provides stimulation to the patient via the dual-purpose electrodes 56. In this state, the dual-purpose electrodes 56 are in their stimulating state of operation, wherein the controller connects the dual-purpose electrodes 56 to the stimulation generator, either simultaneously or sequentially.

The active electrode 54 comprises a wand 60 adapted to be held by the user. The wand is connected to the housing via a cable 62 attached at a distal end thereof, and comprises a metallic tip 64, electrically connected with the cable 62, at a proximal end thereof. A retractable tip cover to may be provided, so that the tip 64 may be protected when the device 50 is not in use. Also, a slide (not shown) may provided to control the tip cover. The slide may be spring-loaded, in order to ensure that the tip cover remains closed when the device 50 is not in use. In addition, indicator lights 68 are provided, for example to indicate power or operative state of the device, whether the active electrode 54 is ready to indicate an error, etc. The dual-purpose electrodes 56 may be any known type of electrode, such as a pad detachably attachable to a patient's skin. They may each be attached to the housing 52 via an individual cable, as shown, or two cables attached to the housing may branch into four cable ends, to each of which a dual-purpose electrode is connected. The dual-purpose electrodes are adapted, during the first operative state of the device 50, to be passive, i.e., to be electrically grounded, and during the second operative state of the device to be active, i.e., to provide the stimulation, as described above.

The user interface 58 comprises buttons 58a, indicator lights 58b, and a speaker 58c. The buttons 58a may be used, for example, to turn the device 50 on and off, to reset the device, such as in the event of an error, and/or to start a new diagnostic test. The indicator lights 58b may indicate the operative state or general state (i.e., warming up, resetting, etc.) of the device 50, whether or not it is on, or if an error has occurred. The speaker 58c is adapted to provide audible instructions to the user, such as to announce the next BAP to which an electrical signal should be applied (this will become clear below in the discussion of the use of the device), if and what type of error has occurred, etc.

The processing unit is adapted to control the overall functionality of the device 50, such as controlling the timing and strength of electrical signals via the electrical signal generator, determining the operative state of the device via the controller, regulating the strength of the stimulation, controlling announcements via the speaker 58c, etc. In addition, it draws diagnostic conclusions from the measurements taken, and is adapted to output them through the data port, for example to a computer, for display on a monitor etc. This may be accomplished using the device of the present invention and partially employing the methods described in U.S. Pat. No. 6,934,581 and/or in WO 2005/117522.

In operation, when a diagnostic test is to be preformed for a patient of his biological systems using the diagnostic device 50, the hand positioning member 30 and the foot positioning member 40 are fastened to the wrist 10 and foot 20 respectively to assume the position described with respect to FIGS. 5A and 5B. Four dual-purpose electrodes 56 are then connected to stimulation points LS1, LS2, RS1 and RS2, corresponding to BAPs GI 4 and E36 on each hand and leg respectively.

Once the positioning members 30, 40 and the dual-purpose electrodes 56 are in position, the active electrode 54 of the diagnostic device 50 is successively brought into contact with each of the measurement points LH1 to LH4, LF1 to LF4, RH1 to RH4, RF1 to RF4 on both hands 10 and feet 20 through the positioning holes 34, 44. This provides a first set of readings according to sixteen samples altogether—four from each hand and four from each foot. For the purpose of measurement, an electrical signal of, e.g. about 15 μA, is sent from the active electrode 54 to the measurement point, and the electrical resistance is measured between the measurement point the dual-purpose electrode 56 attached to stimulation points on the other side of the body serves as the ground. For example, when measuring points LH1 to LH4 (BAPs on the left hand), the dual purpose electrode 56 connected to RS1 (BAP GI 4 on the right hand) serves as ground.

After getting sixteen reading from the sixteen measurement points, appropriate software found either on the device 50 or on a computer performs a calculation of the readings and constructs the normal corridor as described in the Background of the invention. After construction of the normal corridor, each measurement is compared to the corridor to see if it falls inside or outside thereof.

The dual-purpose electrodes 56 then perform stimulation of the stimulation points by applying an electrical signal thereto. The purpose of the electrical signal is to stimulate the entire body. It is known to provide stimulation to the entire body by stimulating only the stimulation points LS1, LS2, RS1 and RS2, therefore the dual-purpose electrodes are placed there. The stimulation may be between 0 and 25 mA, and may be in the form of a pulsed current of a frequency substantially in the range of 75 to 125 Hz, for example substantially 100 Hz. The pulses may be rectangular pulses, and may have a voltage in the range of 3 to 5 volts.

After the aforementioned stimulation is over, a second set of sixteen readings is taken the same way as the first set. Computer software then analyzes the second set by comparing whether or not each reading falls inside or outside the normal corridor previously constructed.

If both a specific result from the first set of measurements (i.e. before applying stimulation) and a corresponding result from the second set of measurements (i.e. after stimulating) fall outside the normal corridor, these two specific results indicate the presence of an imbalance in the related system. However, if one of the results in one of the two sets of measurements falls inside the normal corridor, and the corresponding result from the other set of measurements falls outside the universal corridor, it is considered a false imbalance indication and is therefore disregarded.

Furthermore, if both measurements fall above or below the normal corridor, software may determine that the corresponding system suffers from a certain imbalance.

Analysis of measurements may be, for example as specified below: where the following parametric representation is used:

λ—Midline according to which the normal corridor was constructed;

ε⁺—Deviation above the upper border of the normal corridor;

ε⁺—Deviation below the lower border of the normal corridor;

α—Unilateral deviation from the normal corridor;

β—Bi-lateral deviation from the normal corridor; and

Φ—Degree of functioning of an internal system.

The degree of functioning of a system may be determined, for example, as follows:

Immune System (I)—Average Value and Deviation of RP3 Left:

4 μA<λ: ε⁺<1 μA=>Φ=80%

4 μA<λ: 1 μA<ε⁺<2 μA=>Φ=70%

4 μA<λ: 2 μA<ε⁺=>Φ=60%

4 μA<λ: ε⁻<1 μA=>Φ=90%

4 μA<λ: 2 μA<ε⁻<2 μA=>Φ=85%

4 μA<λ: 2 μA<ε⁻=>Φ=80%

4 μA<λ: ε⁺<1 μA=>Φ=70%

4 μA<λ: 1 μA<ε⁺<2 μA=>Φ=60%

4 μA<λ: 2 μA<ε⁺=>Φ=55%

4 μA<λ: ε⁻<1 μA=>Φ=80%

4 μA<λ: 2 μA<ε⁻<2 μA=>Φ=75%

4 μA<λ: 2 μA<ε⁻=>Φ=80%

Urinary-Genital System (U)—BAP V65 Deviation:

ε⁺<1 μA=>Φ=70%

ε⁺<1 μA=>Φ=60%

ε⁻<1 μA=>Φ=85%

ε⁻<1 μA=>Φ=80%

Alternative=>Φ=55%

A similar analysis may be used for evaluation of the remaining systems such as Cardiovascular, Respiratory, Digestive, and Endocrine, using appropriate parameters and algorithm.

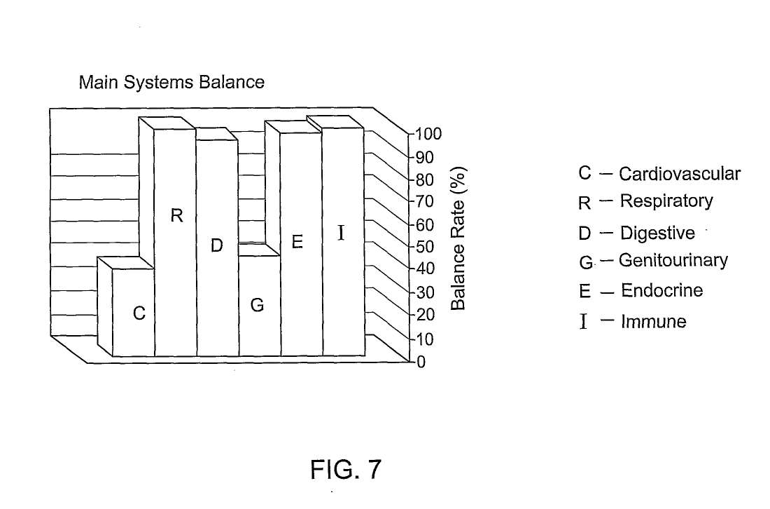

Based on the above analysis, the device may output a system status report, indicating the function of various biological systems within the patient's body. An example of such a report is shown in FIG. 7.

The report according to FIG. 7 gives an output for the balance of six systems: Cardiovascular, Respiratory, Digestive, Genitourinary, Endocrine and Immune, generally designated C, R, D, G, E and I respectively. Each of the systems is rated for balance on a scale of 0-100 (%). In the present example, it is obvious that the C and G systems are at an imbalance, reaching only 32% and 40% respectively. According to this output, the software may recommend a number of food supplements and vitamins that may potentially raise the balance levels of these systems, for example:
For Cardiovascular system ‘C’—Vitamin B6+Vitamin E+Folic Acid+Magnesium.
For Genitourinary system ‘G’—Neutral drugs, for example “Life Stream”+“CoQ10”+“Performa”, “Sunrider” etc.

Based on the above output, decisions may be made and recommendation given on food supplements or vitamins which the patient may need.

The method and positioning member according to the present invention allow a quick (i.e. several minutes), non-intrusive and simple process, for determining a system imbalance at a patient, and providing appropriate food supplements and vitamins for at least partially restoring balance to the imbalanced systems.

Those skilled in the art to which this invention pertains will readily appreciate that numerous changes, variations, and modifications can be made without departing from the scope of the invention, mutatis mutandis.

1-41. (canceled)

42. An internal systems imbalance determining method for use in the determination of suitable food supplements and vitamins provision for, and in the control of their usage by, a patient; the patient having on his skin at least one sensing zone with at least one measurement point located therein, and at least one stimulation point, the method comprising:
providing a diagnostic device for measuring electrical resistance between at least one measurement point on one side of the patient's body, and a ground point on another side of the body which is electrically connected to ground, by applying electrical signal to the at least one measurement point, the device comprising an active electrode adapted to provide the electrical signal by coming in contact with the at least one measurement point, and at least one dual-purpose electrode adapted to selectively provide electrical ground or electrical stimulation when in contact with a corresponding ground or stimulation point on the patient's skin;
providing at least one positioning member adapted to be placed over and at least partially cover the at least one sensing zone of the patient, and formed with at least one positioning hole adapted to coincide with the at least one measurement point, when the positioning member is placed over the at least one sensing zone, so as to allocate the at least one measurement point and allow the active electrode to come in contact therewith through the at least one positioning hole;
placing the positioning member on the sensing zone on one side of the body;
bringing the active electrode in contact with the measurement point through the positioning hole, to provide the electrical signal thereto, and providing electrical ground at the ground point by the dual-purpose electrode, and collecting a first measurement therefrom;
removing the active electrode and providing electrical stimulation at the stimulation point using the dual-purpose electrode;
repeating bringing the active electrode in contact with the measurement point through the positioning hole, to provide the electrical signal thereto, and providing electrical ground at the ground point by the dual-purpose electrode, and collecting a first measurement therefrom for collecting a second measurement from the measurement point;
performing analysis of the first and the second measurements thereby allocating possible imbalance of internal systems of the body; and
outputting results of the analysis in a manner suitable, or presenting recommendations suitable for selecting at least one food supplement and/or vitamin, based on the analysis.

43. The method according to claim 42, wherein the measurement is taken from one of the following points: P9, MC7, C7, GI5, RP3, F3, V65 and E42, corresponding to the internal systems of the body.

44. The method according to claim 42, wherein the electrical signal provided by the active electrode for the purpose of the measurement is between 0 and 20 μA.

45. The method according to claim 42, wherein the diagnostic device is adapted to provide an assessment of the condition of internal systems in order to determine body imbalance based on a plurality of the measurements.

46. The method according to claim 45, wherein the internal systems are one or more of the following: Immune, Endocrine, Respiratory, Cardiovascular, Digestive and Genital-Urinary.

47. The method according to claim 42, wherein the dual purpose electrodes are adapted for attachment to at least one of four stimulating points, selected from the group consisting of: E36 on the left leg, E36 on the right leg, GI 4 on the left arm, and GI 4 on the right arm.

48. The method according to claim 47, wherein one of the dual-purpose electrodes is adapted, in its stimulating mode, to apply stimulation between 0 and 25 mA.

49. The method according to claim 48, wherein the stimulation is in the form of a pulsed current of a frequency in the range of from about 75 to about 125 Hz.

50. The method according to claim 49, wherein the pulses are rectangular pulses, and have a voltage in the range of 3 to 5 volts.

51. A positioning member for use with a diagnostic device having an electrode, and adapted for mounting on a patient, the patient having on his skin at least one sensing zone with at least one measurement point located therein, the positioning member being adapted to be placed over and at least partially cover the sensing zone, and being formed with at least one positioning hole adapted to coincide with the at least one measurement point, when the positioning member is placed over the sensing zone, so as to allocate the at least one measurement point and allow the electrode to come in contact therewith through the at least one positioning hole.

52. The positioning member according to claim 51, which is in the form of a strap, band, or the like, adapted to be placed on a wrist or a foot of the patient.

53. The positioning member according to claim 52, adapted for mounting on a left foot, a right foot, a left hand or a right hand.

54. The positioning member according to claim 52, adapted to switch between a left and a right orientation by inversion of the positioning member.

55. The positioning member according to claim 54, wherein the switch between a left and a right orientation is achieved by inversion of the positioning member.

56. The positioning member according to claim 51, wherein the member is constructed so as not to bias measurements taken by the sensor.

57. The positioning member according to claim 51, wherein the positioning hole is marked, or has an indicia so as to indicate the appropriate BAP to be measured.

58. The positioning member according to claim 57, wherein according to the indicia, an operator of the electrode knows the order of BAP's to be measured, as well as to associate the measurement acquired with the appropriate BAP.

59. A package comprising a plurality of positioning members for use with a diagnostic device, adapted for mounting on a patient having on his skin at least one sensing zone with at least one measurement point located therein, each of the plurality of positioning members being adapted to be placed over and at least partially cover the sensing zone, and being formed with at least one positioning hole adapted to coincide with the at least one measurement point, when the positioning member is placed over the sensing zone, so as to allocate the at least one measurement point and allow an active electrode to come in contact therewith through the at least one positioning hole.

60. The package according to claim 59 comprising positioning member of same orientation.

61. The package according to claim 59, wherein the package is a kit comprising positioning members of various orientations, adapted for use of a single person.
